(19) United States
(12) Patent Application Publication
(10) Pub. No.: US 2026/0209700 A1
(43) Pub. Date: Jul. 23, 2026

(54) COMPOSITION AND METHOD FOR EX VIVO EXPANSION OF STEM CELL MEMORY T CELLS

(71) Applicant: LUKAS BIOMEDICAL INC., New Taipei City (TW)

(72) Inventors: Wei Hua CHU, Kaohsiung City (TW); Bin HSU, New Taipei City (TW); Yi-Jia TSAI, New Taipei City (TW)

(21) Appl. No.: 19/225,684

(22) Filed: Jun. 2, 2025

(30) Foreign Application Priority Data
Jan. 17, 2025 (TW) 114102106

(51) Int. Cl. C12N 5/0783

(52) U.S. Cl. CPC C12N 5/0636; C12N 2501/2302; C12N 2501/2307; C12N 2501/2315; C12N 2501/51; C12N 2501/515; C12N 2501/999; C12N 2502/115

(57) ABSTRACT

The present disclosure provides a composition for ex vivo expansion of stem cell memory T cells, comprising: a first composition, which includes CD3, CD28, interleukin-2 (IL-2), interleukin-7 (IL-7) and interleukin-15 (IL-15); and a second composition, which includes interleukin-2, interleukin-7, and interleukin-15. The present disclosure also provides a method for ex vivo expansion of stem cell memory T cells, comprising: culturing mononuclear cells in a medium containing the first composition to obtain activated T cells; culturing the activated T cells in a medium containing the second composition to obtain a first cell population; and culturing the obtained first cell population in a medium containing a third composition to obtain a second cell population. Therefore, the composition and method for ex vivo expansion of the stem cell memory T cells provided by the present disclosure may be applied in clinical fields related to immunotherapy.

### CROSS-REFERENCE TO RELATED APPLICATIONS

This non-provisional application claims priority under 35 U.S.C. § 119 (a) on Patent Application No(s). 114102106 filed in Taiwan on Jan. 17, 2025, the entire contents of which are hereby incorporated by reference.

### BACKGROUND

### 1. Technical Field

This disclosure relates to a composition and method for ex vivo expansion of stem cell memory T cells.

### 2. Related Art

Stem cell memory T cells (Tscm) play a critical role in the immune system due to their ability to long-term maintain immune memory and rapidly proliferate T cells upon antigen re-exposure, thereby initiating swift immune responses. Compared to memory cells at other stages, stem cell memory T cells exhibit superior therapeutic efficacy in cancer treatment, demonstrating significant potential in disease therapies, particularly for immune-related conditions such as cancer and chronic infections.

In prior art, the efficiency and stability of large-scale expansion of stem cell memory T cells populations remain quite limited, and it is also difficult to control the proportions of various subpopulations in the resulting cells. Therefore, there is an urgent need for providing a novel composition and method to more effectively increase the yield of stem cell memory T cells while simultaneously maintaining their immune functions.

### SUMMARY

Accordingly, an embodiment of the present disclosure provides a composition and method for ex vivo expansion of CD3+CD45RO−CD197+CD95+ stem cell memory T cells, which may significantly enhance the cell expansion fold of stem cell memory T cells.

An embodiment of the present disclosure provides a composition for ex vivo expansion of memory T stem cells, comprising:
a first composition comprising CD3, CD28, interleukin-2 (IL-2), interleukin-7 (IL-7) and interleukin-15 (IL-15); and
a second composition comprising interleukin-2, interleukin-7 and interleukin-15.

Another embodiment of the present disclosure provides a method for ex vivo expansion of stem cell memory T cells, comprising:
culturing mononuclear cells in a medium containing the first composition to obtain activated T cells;
culturing the activated T cells in a medium containing the second composition to obtain a first cell population; and
culturing the first cell population in a medium containing a third composition to obtain a second cell population.

The composition and method for ex vivo expansion of stem cell memory T cells according to an embodiment of the present disclosure, the resulting cell population comprises a high proportion of stem cell memory T cells. Also, compared to the cell populations produced using compositions disclosed in the prior art, the first composition of the present disclosure effectively activates mononuclear cells to obtain activated T cells; culturing the activated T cells obtained using the first composition continued using the second composition obtains a first cell population; and further culturing the first cell population using the third composition obtains a second cell population. Both the first and second cell populations exhibit significantly increased expansion folds of stem cell memory T cells and central memory T cells compared to comparative examples, with the cell expansion fold of the stem cell memory T cells being especially pronounced. Furthermore, the cell populations produced using the composition and method for ex vivo expansion of memory T stem cells according to the embodiments of the present disclosure also include a high proportion of activated T cells, natural killer T cells, and cytotoxic T cells. Accordingly, the composition and method for ex vivo expansion of memory T stem cells developed in the present disclosure may significantly enhance the cell expansion fold of stem cell memory T cells while concurrently endowing the cells with functional cell surface proteins. Stem cell memory T cells possess a prolonged lifespan and excellent self-renewal capability, enabling rapid differentiation and robust immune responses upon antigenic stimulation. This greatly enhances the potential of cell therapy and renders them an ideal candidate cell source for cancer immunotherapy.

In addition, the high-quality stem cell memory T cells produced by the composition and method of the present disclosure may serve as a critical raw material for the manufacture of chimeric antigen receptor T cells (CAR-T cells). Due to their excellent proliferative capacity and durability, these cells are anticipated to enhance the persistence of CAR-T cells in clinical treatments, thereby improving therapeutic efficacy.

### BRIEF DESCRIPTION OF THE DRAWINGS

The present disclosure will become more fully understood from the detailed description given hereinbelow and the accompanying drawings which are given by way of illustration only and thus are not limitative of the present disclosure and wherein:

FIG. 1 is a flowchart illustrating a method for obtaining ex vivo expansion of stem cell memory T cells using the composition for ex vivo expansion of memory T stem cells of the present disclosure.

FIG. 2 illustrates the relationship between a cell expansion fold of a first cell population obtained using Embodiments 1 to 4 and Comparative Example 1 during a process of obtaining the first cell population.

FIG. 3 illustrates the relationship between the cell expansion fold of the first cell population obtained using Embodiments 5 to 9 and Comparative Example 1 during the process of obtaining the first cell population.

FIG. 4 illustrates the relationship between the cell expansion fold of the second cell population obtained using Embodiments 10 to 13 and Comparative Example 3 during a process of obtaining the second cell population.

FIG. 5 illustrates the relationship between the cell expansion fold of the second cell population obtained using Embodiments 14 to 18 and Comparative Example 2 during the process of obtaining the second cell population.

FIG. 6 illustrates an analysis of proportions of activated T cells, natural killer T cells, and cytotoxic T cells in the cell population obtained by culturing Embodiments 10 to 13 and Comparative Example 3 of the present disclosure during the process of obtaining the second cell population.

FIG. 7 illustrates an analysis of the proportions of activated T cells, natural killer T cells, and cytotoxic T cells in the cell population obtained by culturing Embodiments 14 to 18 and Comparative Example 2 of the present disclosure during the process of obtaining the second cell population.

FIG. 8 illustrates an analysis after consolidating data from various batches of the proportions of activated T cells, natural killer T cells, and cytotoxic T cells in the cell population obtained by culturing Embodiments 10 to 18 and Comparative Examples 2 and 3 of the present disclosure during the process of obtaining the second cell population.

FIG. 9 is a diagram illustrating a flow cytometric analysis of the cells obtained by culturing using Embodiment 11 of the present disclosure during the process of obtaining the second cell population.

FIG. 10 is a diagram illustrating a flow cytometric analysis of the cells obtained by culturing using Comparative Example 2 of the present disclosure during the process of obtaining the second cell population.

### DETAILED DESCRIPTION

In the following detailed description, for purpose of explanation, numerous specific details are set forth in order to provide a thorough understanding of the disclosed embodiments. It will be apparent, however, that one or more embodiments may be practiced without these specific details. In other instances, well-known structures and devices are schematically shown in order to simplify the drawing.

An embodiment of the present disclosure provides a composition for ex vivo expansion of memory T stem cells, including:
a first composition including CD3, CD28, interleukin-2 (IL-2), interleukin-7 (IL-7) and interleukin-15 (IL-15); and
a second composition including interleukin-2, interleukin-7 and interleukin-15.

In an embodiment of the present disclosure, the concentration of CD3 may be 100 ng/mL to 800 ng/mL. In other embodiments, the concentration of CD3 may be 10 ng/ml to 1600 ng/mL. In other embodiments, the concentration of CD3 may be 200 ng/mL to 400 ng/mL. In other embodiments, the concentration of CD3 may be 100 ng/ml to 200 ng/mL.

In an embodiment of the present disclosure, the concentration of CD28 may be 300 ng/mL to 2000 ng/mL. In other embodiments, the concentration of CD28 may be 30 ng/ml to 4000 ng/mL. In other embodiments, the concentration of CD28 may be 600 ng/ml to 1200 ng/mL. In other embodiments, the concentration of CD28 may be 300 ng/ml to 600 ng/mL.

Based on common unit conversions between activity units and weight units for interleukin-2 available on the market, 1 gram (g) of protein may correspond to 6.1 activity units (U) to 10 activity units. For example, when using a conversion formula where 1 ng=10 U provided by Peprotech, the content of interleukin-2 in the composition for ex vivo expansion of memory T stem cells of the present disclosure is 5 ng/ml to 25 ng/mL; and when using a conversion formula where 1 ng=6.1 U provided by Miltenyi, the content of interleukin-2 in the composition is 8.2 ng/mL to 41 ng/mL. Accordingly, in the composition for ex vivo expansion of memory T stem cells of the present disclosure, the content of interleukin-2 may be 5 ng/mL to 41 ng/mL, but is not limited thereto. In other embodiments, the content of interleukin-2 may be 0.5 ng/ml to 30 ng/mL. In other embodiments, the content of interleukin-2 may be 0.820 ng/mL to 49.2 ng/mL. In other embodiments, the content of interleukin-2 may be 10 ng/mL to 15 ng/mL. In other embodiments, the content of interleukin-2 may be 16.4 ng/ml to 24.6 ng/mL. In other embodiments, the content of interleukin-2 may be 5 ng/mL to 10 ng/mL. In other embodiments, the content of interleukin-2 may be 8.2 ng/ml to 16.4 ng/ml.

Based on common unit conversions between activity units and weight units for interleukin-7 available on the market, 1 gram (g) of protein may corresponds to 2 activity units (U) to 130 activity units. For example, when using a conversion formula where 1 ng=2 U provided by Peprotech, the content of interleukin-7 in the composition for ex vivo expansion of memory T stem cells of the present disclosure is 12.5 ng/ml to 25 ng/ml, and when using a conversion formula where 1 ng=130 U provided by Miltenyi, the content of interleukin-7 in the composition is 0.192 ng/mL to 0.385 ng/mL. Accordingly, in the composition for ex vivo expansion of memory T stem cells of the present disclosure, the content of interleukin-7 may be 0.192 ng/mL to 25 ng/mL, but is not limited thereto. In other embodiments, the content of interleukin-7 may be 0.077 ng/ml to 0.769 ng/mL.

Based on common unit conversions between activity units and weight units for interleukin-15 available on the market, 1 gram (g) of protein may corresponds to 2 activity units (U) to 20 activity units. For example, when using a conversion formula where 1 ng=2 U provided by Peprotech, the content of interleukin-15 in the composition for ex vivo expansion of memory T stem cells of the present disclosure is 5 ng/ml to 25 ng/ml, and when using a conversion formula where 1 ng=20 U provided by Miltenyi, the content of interleukin-15 in the composition is 0.5 ng/mL to 2.5 ng/mL. Accordingly, in the composition for ex vivo expansion of memory T stem cells of the present disclosure, the content of interleukin-15 may be 0.5 ng/mL to 25 ng/mL, but is not limited thereto. In other embodiments, the content of interleukin-15 may be 12.5 ng/ml to 25 ng/ml. In other embodiments, the content of interleukin-15 may be 1.25 ng/ml to 2.5 ng/mL.

In other embodiments, the first composition includes 100 ng/ml to 800 ng/ml or 100 ng/mL to 200 ng/mL of CD3, 300 ng/mL to 2000 ng/ml or 300 ng/ml to 600 ng/ml of CD28, 50 U/mL to 250 U/mL of interleukin-2, 25 U/mL to 50 U/mL of interleukin-7, and 25 U/mL to 50 U/mL of interleukin-15.

In other embodiments, the second composition includes 100 U/mL of interleukin-2, 50 U/mL of interleukin-7, and 50 U/mL of interleukin-15.

In other embodiments, the third composition includes from 25 U/mL to 50 U/mL of interleukin-7, from 10 U/mL to 50 U/mL of interleukin-15, from 1 mM to 10 mM of N-acetylcysteine, and from 0% to 5% (v/v) of human platelet lysate.

Another embodiment of the present disclosure provides a method for ex vivo expansion of memory T stem cells, including: culturing mononuclear cells in a medium containing the first composition to obtain activated T cells, culturing the activated T cells in a medium containing the second composition to obtain a first cell population, and culturing the first cell population in a medium containing the third composition to obtain a second cell population. The activated T cells are CD3+ activated T cells. The first cell population includes CD3+CD45RO−CD197+CD95+ stem cell memory T cells, CD3+CD45RO+CD197+ central memory T cells, CD3+CD45RO+CD197− effector memory T cells, CD3+CD56+ natural killer T cells, and CD3+CD8+ cytotoxic T cells. The second cell population includes CD3+ activated T cells, CD3+CD45RO−CD197+CD95+ stem cell memory T, CD3+CD45RO+CD197+ central memory T cells, CD3+CD45RO+CD197− effector memory T cells, CD3+CD56+ natural killer T cells, and CD3+CD8+ cytotoxic T cells.

In the following, CD3+ activated T cells are referred to as “activated T cells”, CD3+CD45RO−CD197+CD95+ stem cell memory T cells will be referred to as “stem cell memory T cells”, CD3+CD45RO+CD197+ central memory T cells are referred to as “central memory T cells”, CD3+CD45RO+CD197− effector memory T cells are referred to as “effector memory T cells”, CD3+CD56+ natural killer T cells are referred to as “natural killer T cells”, and CD3+CD8+ cytotoxic T cells are referred to as “cytotoxic T cells”.

The activated T cells may be obtained by culturing the mononuclear cells in the medium containing the first composition of the present disclosure. The mononuclear cells may be derived from peripheral blood, umbilical cord blood, or bone marrow blood; however, the present disclosure is not limited thereto. For example, first, peripheral blood is obtained and diluted with phosphate-buffered saline (PBS), and is isolated and purified by using a cell separation medium (Ficoll-Paque) to obtain the mononuclear cells. Next, the peripheral blood mononuclear cells are cultured using a basic culture medium which may include, but is not limited to, DMEM, X-VIVO, TexMACS medium, RPMI-1640, or AIM-V, and CD3, CD28, interleukin-2 (IL-2), interleukin-7 (IL-7), and interleukin-15 (IL-15) are added to activate the T cells. A culture time for the mononuclear cells may be 2 to 4 days, a culture temperature may be 37° C., and the air environment may be atmosphere+5% CO₂. In this way, the activated T cells may be obtained by culturing the mononuclear cells.

The first cell population may be obtained by culturing the activated T cells in the medium containing the second composition of the present disclosure. The first cell population includes the activated T cells, the stem cell memory T cells, the central memory T cells, and the effector memory T cells. The culture time for the first cell population may be 4 to 6 days, the culture temperature may be 37° C., and the air environment may be atmosphere+5% CO₂.

The second cell population may be obtained by culturing the first cell population in the medium containing the third composition of the present disclosure. The culture time for the second cell population may be 3 to 6 days, the culture temperature may be 37° C., and the air environment may be atmosphere+5% CO₂.

Thus, the resulting second cell population may include the activated T cells, the stem cell memory T cells, the central memory T cells, the effector memory T cells, the natural killer T cells, and the cytotoxic T cells.

As described above, the first cell population and the second cell population obtained by culturing the composition for ex vivo expansion of memory T stem cells of the present disclosure, as compared to the comparative examples, may significantly enhance the cell expansion fold of stem cell memory T cells while concurrently producing the T cell populations with functional cell surface proteins.

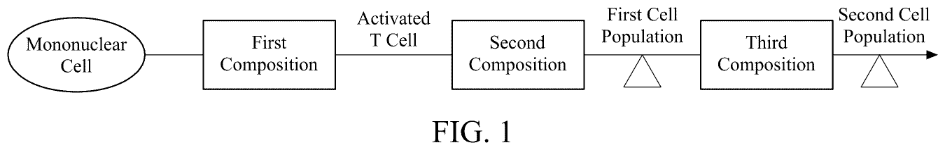

The following experiments use spectral flow cytometry to analyze the proportions of various cell populations in the first cell population and the second cell population. In detail, referring to FIG. 1, first, the mononuclear cells are cultured using the first composition of each embodiment or comparative example for 2 to 4 days to obtain the activated T cells. Next, the activated T cells are cultured using the second composition of each embodiment or comparative example for 4 to 6 days to obtain the first cell population. Next, the first cell population is cultured using the third composition of each embodiment or comparative example for 3 to 6 days to obtain the second cell population. A required number of cells are then taken from the first population and the second cell population and washed with phosphate-buffered saline. Thereafter, fluorescent antibodies are added for cell staining. The cells are then washed twice with phosphate-buffered saline and analyzed by a spectral flow cytometer to determine the proportions of various cell populations.

### [Experiment 1. The Relationship Between Different Concentrations of CD3 and CD28 in the First Composition and the Cell Expansion Fold of the First Cell Population]

In Embodiments 1 to 4, the mononuclear cells are cultured in a basal medium supplemented with the first composition, which respectively includes 100 to 800 ng/ml of CD3, 300 to 2000 ng/ml of CD28, 100 U/mL of IL-2, 50 U/mL of IL-7, and 50 U/mL of IL-15. The mononuclear cells are then cultured for 3 days to obtain the activated T cells. Next, the activated T cells are cultured in a basal medium supplemented with the second composition, which includes 100 U/mL of IL-2, 50 U/mL of IL-7, and 50 U/mL of IL-15. The activated T cells are then cultured for 6 days to obtain the first cell population. The cell expansion fold of the first cell population is then calculated and analyzed. Table 1 shows that the first compositions in Embodiments 1 to 4 have different concentrations of CD3 and CD28 and fixed concentrations of IL-2, IL-7, and IL-15.

For comparison, Comparative Example 1, which employs a two-stage cell culture method, is provided:
First stage (Day 0 to Day 5): mononuclear cells are cultured in X-Vivo basal medium supplemented with 50 μg/mL gentamicin, 5% human serum, 220 U/mL of IL-2, and 5 μg/mL of CD3 to obtain an intermediate cell product.
Second stage (Day 6 to Day 7): The intermediate cell product is further cultured in X-Vivo basal medium supplemented with 50 μg/mL gentamicin, 5% human serum, and 220 U/mL of IL-2 to obtain the target cell population. The cell expansion fold of the obtained cell population is then calculated and analyzed.

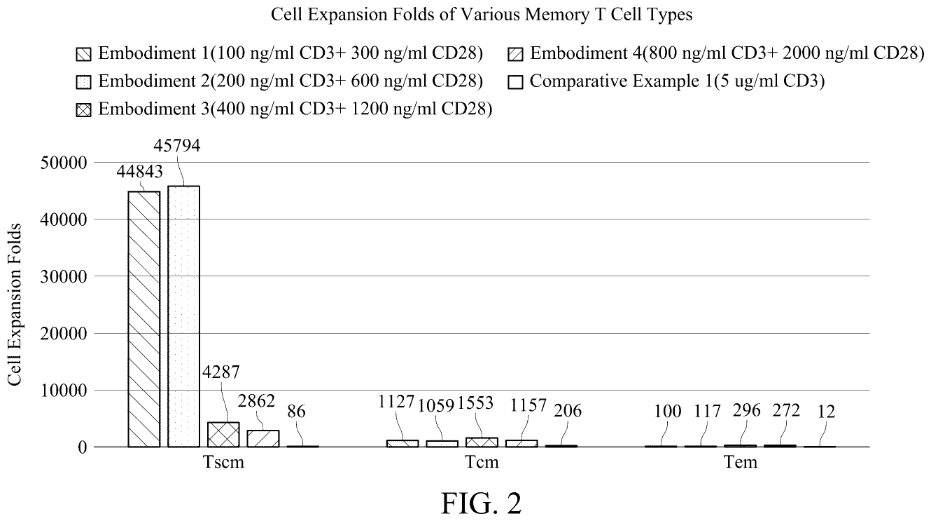

FIG. 2 and Table 2 illustrate the cell expansion folds of stem cell memory T cells (Tscm), central memory T cells (Tcm), and effector memory T cells (Tem) obtained using Embodiments 1 to 4 and Comparative Example 1.

Calculation method for cell expansion fold is as follows:

$$1. Total cell number \times Cell proportion of each cell type = Number of$$

$$each cell type$$

$$2. Number of each cell type / Number of each cell type on Day 0$$

$$( mononuclear cells ) = Cell expansion fold of each cell type .$$

As shown in FIG. 2 and Table 2, after culturing the cells in the basal medium supplemented with the first composition of Embodiments 1 to 4, the results demonstrate that 5 the stem cell memory T cells (Tscm), the central memory T cells (Tcm), and the effector memory T cells (Tem) all exhibit higher cell expansion folds compared to Comparative Example 1, wherein the cell expansion fold of the stem cell memory T cells (Tscm) is dramatically increased (greater than 86-fold) compared to Comparative Example 1.

TABLE 1
The concentrations of CD3, CD28, IL-2, IL-7, and IL-15
in the first composition, and the concentrations of
IL-2, IL-7, and IL-15 in the second composition

| | | Embodi- | Embodi- | Embodi- | | Embodi- | |
|---|---|---|---|---|---|---|---|
| | | ment 1 | ment 2 | ment 3 | | ment 4 | |
| First | CD3 | 100 ng/ml | 200 ng/ml | 400 | ng/ml | 800 | ng/ml |
| Composition | CD28 | 300 ng/ml | 600 ng/ml | 1200 | ng/ml | 2000 | ng/ml |

| | | |
|---|---|---|
| | IL-2 | 100 U/ml |
| | IL-7 | 50 U/ml |
| | IL-15 | 50 U/ml |
| Second | IL-2 | 100 U/ml |
| Composition | IL-7 | 50 U/ml |
| | IL-15 | 50 U/ml |

TABLE 2
Cell expansion folds of various memory T cell types

| | Tscm | Tcm | Tem |
|---|---|---|---|
| Embodiment 1 | 44843 | 1127 | 100 |
| Embodiment 2 | 45794 | 1059 | 117 |
| Embodiment 3 | 4287 | 1553 | 296 |
| Embodiment 4 | 2862 | 1157 | 272 |
| Comparative | 86 | 206 | 12 |
| Example 1 | | | |

### [Experiment 2. The Relationship Between Different Concentrations of IL-2, IL-7, and IL-15 in the First Composition and the Cell Expansion Fold of the First Cell Population]

In Embodiments 5 to 9, the mononuclear cells are cultured in a basal medium supplemented with the first composition, which respectively includes 200 ng/mL of CD3, 600 ng/ml of CD28, 50 U/ml to 250 U/ml of IL-2, 25 U/mL to 50 U/mL of IL-7, and 25 U/mL to 50 U/mL of IL-15. The mononuclear cells are then cultured for 3 days to obtain the activated T cells. Next, the activated T cells are cultured in a basal medium supplemented with the second composition, which includes 100 U/mL of IL-2, 50 U/mL of IL-7, and 50 U/mL of IL-15. The activated T cells are then cultured for 6 days to obtain the first cell population. The cell expansion fold of the first cell population is then calculated and analyzed. Table 3 shows that the first compositions in Embodiments 5 to 9 have fixed concentrations of CD3 and CD28 and different concentrations of IL-2, IL-7, and IL-15. Comparative Example 1 is also used in Experiment 2. As described in Experiment 1, Comparative Example 1 is also employed in Experiment 2 for cell culture.

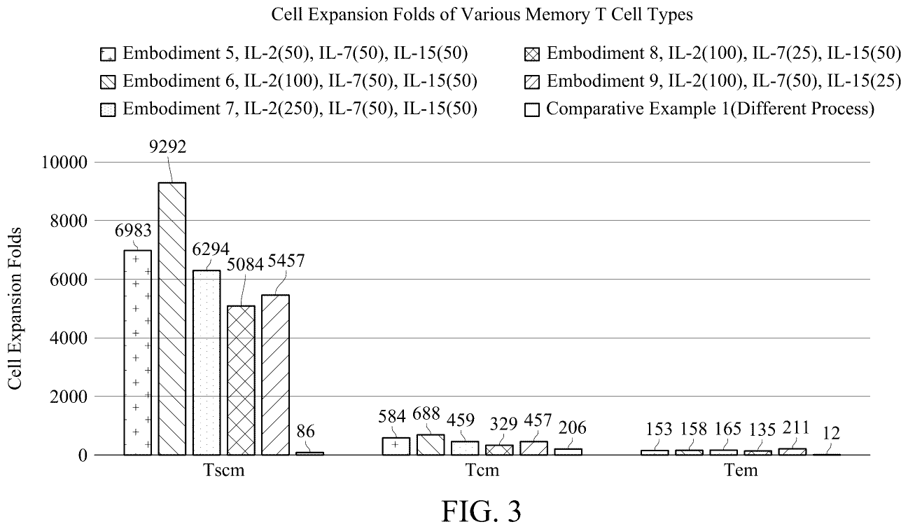

FIG. 3 and Table 4 show the cell expansion folds of the stem cell memory T cells (Tscm), the central memory T cells (Tcm), and the effector memory T cells (Tem) obtained using Embodiments 5 to 9 and Comparative Example 1.

As shown in FIG. 3 and Table 4, culturing cells in the basal medium supplemented with the compositions of Embodiment 5 to 9, the results demonstrate that the stem cell memory T cells (Tscm), the central memory T cells (Tcm), and the effector memory T cells (Tem) all exhibit higher cell expansion folds compared to Comparative Example 1, wherein the cell expansion fold of the stem cell memory T cells (Tscm) is dramatically increased (greater than 86-fold) compared to Comparative Example 1.

TABLE 3
The concentrations of CD3, CD28, IL-2, IL-7, and IL-15 in the first composition
and the concentrations of IL-2, IL-7, and IL-15 in the second composition

| | | Embodi- | Embodi- | | Embodi- | | Embodi- | | Embodi- | |
|---|---|---|---|---|---|---|---|---|---|---|
| | | ment 5 | ment 6 | | ment 7 | | ment 8 | | ment 9 | |
| First | CD3 | 200 ng/ml | | | | | | | | |
| Composition | CD28 | 600 ng/ml | | | | | | | | |
| | IL-2 | 50 U/ml | 100 | U/ml | 250 | U/ml | 100 | U/ml | 100 | U/ml |
| | IL-7 | 50 U/ml | 50 | U/ml | 50 | U/ml | 25 | U/ml | 50 | U/ml |
| | IL-15 | 50 U/ml | 50 | U/ml | 50 | U/ml | 50 | U/ml | 25 | U/ml |

| | | |
|---|---|---|
| Second | IL-2 | 100 U/ml |
| Composition | IL-7 | 50 U/ml |
| | IL-15 | 50 U/ml |

TABLE 4
Cell expansion folds of various memory T cell types

| | Tscm | Tcm | Tem |
|---|---|---|---|
| Embodiment 5 | 6983 | 584 | 153 |
| Embodiment 6 | 9292 | 688 | 158 |
| Embodiment 7 | 6294 | 459 | 165 |
| Embodiment 8 | 5084 | 329 | 135 |
| Embodiment 9 | 5457 | 457 | 211 |
| Comparative Example 1 | 86 | 206 | 12 |

### [Experiment 3. The Relationship Between Different Concentrations of CD3 and CD28 in the First Composition and the Cell Expansion Fold of the Second Cell Population]

In Embodiments 10 to 13, the mononuclear cells are cultured in a basal medium supplemented with the first composition, which respectively includes 100 ng/mL to 800 ng/mL of CD3, 300 ng/mL to 2000 ng/ml of CD28, 100 U/ml of IL-2, 50 U/mL of IL-7, and 50 U/mL of IL-15. The mononuclear cells are then cultured for 3 days to obtain the activated
cells. Next, the activated T cells are cultured in a basal medium supplemented with the second composition, which includes 100 U/mL of IL-2, 50 U/mL of IL-7, and 50 U/mL of IL-15. The activated T cells are then cultured for 6 days to obtain the first cell population. Finally, the first cell population is cultured in a basal medium supplemented with the third composition, which includes 10 mM NAC, 5% hPL, 50 U/mL of IL-7, and 50 U/mL of IL-15. The first cell population is then cultured for 5 days to obtain the second cell population. The cell expansion fold of the second cell population is then calculated and analyzed. Table 5 shows the concentrations of CD3, CD28, IL-2, IL-7, and IL-15 in the first composition, the concentrations of IL-2, IL-7, and IL-15 in the second composition, and the concentrations of NAC, hPL, IL-7, and IL-15 in the third composition used in Embodiments 10 to 13.

For comparison, Comparative Example 2 which employs a two-stage cell culture method, is also provided:
First stage (Day 0 to Day 5): mononuclear cells are cultured in X-Vivo basal medium supplemented with 50 μg/mL gentamicin, 5% human serum, 220 U/mL IL-2, and 5 μg/mL CD3 to obtain an intermediate cell product.
Second stage (Day 6 to Day 15): the intermediate cell product is further cultured in X-Vivo basal medium supplemented with 50 g/mL gentamicin, 5% human serum, and 220 U/mL IL-2 to obtain the target cell population. The cell expansion fold of the target cell population is then calculated and analyzed.

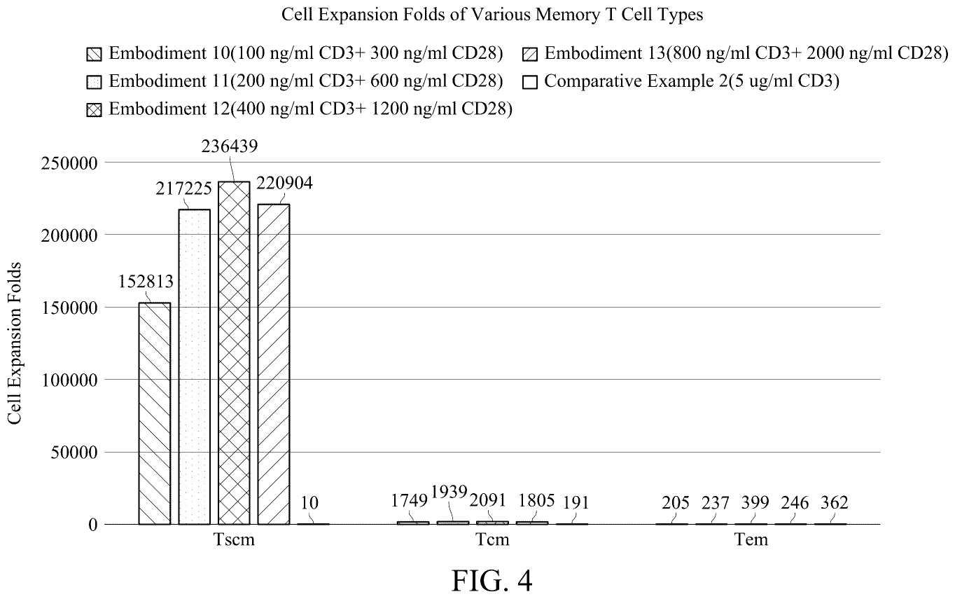

FIG. 4 and Table 5 show the cell expansion folds of the stem cell memory T cells (Tscm), the central memory T cells (Tcm), and the effector memory T cells (Tem) obtained using Embodiments 10 to 13 and Comparative Example 2.

FIG. 4 and Table 6 show that after culturing cells in a basal medium supplemented with the compositions of Embodiments 10 to 13, the results demonstrate that the stem cell memory T cells (Tscm), the central memory T cells (Tcm), and the effector memory T cells (Tem) all exhibit higher cell expansion folds compared to Comparative Example 2, wherein the cell expansion fold of the stem cell memory T cells (Tscm) is dramatically increased (greater than 10-fold) compared to Comparative Example 2.

TABLE 5
The concentrations of CD3, CD28, IL-2, IL-7, and IL-15 in
the first composition, the concentrations of IL-2, IL-7,
and IL-15 in the second composition, and the concentrations
of NAC, hPL, IL-7 and IL-15 in the third composition

| | | Embodi- | Embodi- | Embodi- | | Embodi- | |
|---|---|---|---|---|---|---|---|
| | | ment 10 | ment 11 | ment 12 | | ment 13 | |
| First | CD3 | 100 ng/ml | 200 ng/ml | 400 | ng/ml | 800 | ng/ml |
| Composition | CD28 | 300 ng/ml | 600 ng/ml | 1200 | ng/ml | 2000 | ng/ml |

| | | |
|---|---|---|
| | IL-2 | 100 U/ml |
| | IL-7 | 50 U/ml |
| | IL-15 | 50 U/ml |
| Second | IL-2 | 100 U/ml |
| Composition | IL-7 | 50 U/ml |
| | IL-15 | 50 U/ml |
| Third | NAC | 10 mM |
| Composition | hPL | 5% |
| | IL-7 | 50 U/ml |
| | IL-15 | 50 U/ml |

TABLE 6
Cell expansion folds of various memory T cell types

| | Tscm | Tcm | Tem |
|---|---|---|---|
| Embodiment 10 | 152813 | 1749 | 205 |
| Embodiment 11 | 217225 | 1939 | 237 |
| Embodiment 12 | 236439 | 2091 | 399 |
| Embodiment 13 | 220904 | 1805 | 246 |
| Comparative Example 2 | 10 | 191 | 362 |

### [Experiment 4. The Relationship Between Different Concentrations of IL-2, IL-7, and IL-15 in the First Composition and the Cell Expansion Fold of the Second Cell Population]

In Embodiments 14 to 18, the mononuclear cells are cultured in a basal medium supplemented with the first composition, which respectively includes 200 ng/mL of CD3, 600 ng/ml of CD28, 50 U/ml to 250 U/ml of IL-2, 25 U/ml to 50 U/ml of IL-7, and 25 U/mL to 50 U/mL of IL-15. The mononuclear cells are then cultured for 3 days to obtain the activated T cells. Next, the activated T cells are cultured in a basal medium supplemented with the second composition, which includes 100 U/mL of IL-2, 50 U/mL of IL-7, and 50 U/mL of IL-15. The activated T cells are then cultured for 6 days to obtain the first cell population. Finally, the first cell population is cultured in a basal medium supplemented with the third composition, which includes 10 mM NAC, 5% hPL, 50 U/mL of IL-7, and 50 U/mL of IL-15. The first cell population is then cultured for 5 days to obtain the second cell population. The cell expansion fold of the second cell population is then calculated and analyzed. Table 7 shows the concentrations of CD3, CD28, IL-2, IL-7, and IL-15 in the first composition, the concentrations of IL-2, IL-7, and IL-15 in the second composition, and the concentrations of NAC, hPL, IL-7, and IL-15 in the third composition used in Embodiments 14 to 18. As described in Experiment 3, Comparative Example 2 is also employed in Experiment 4 for cell culture.

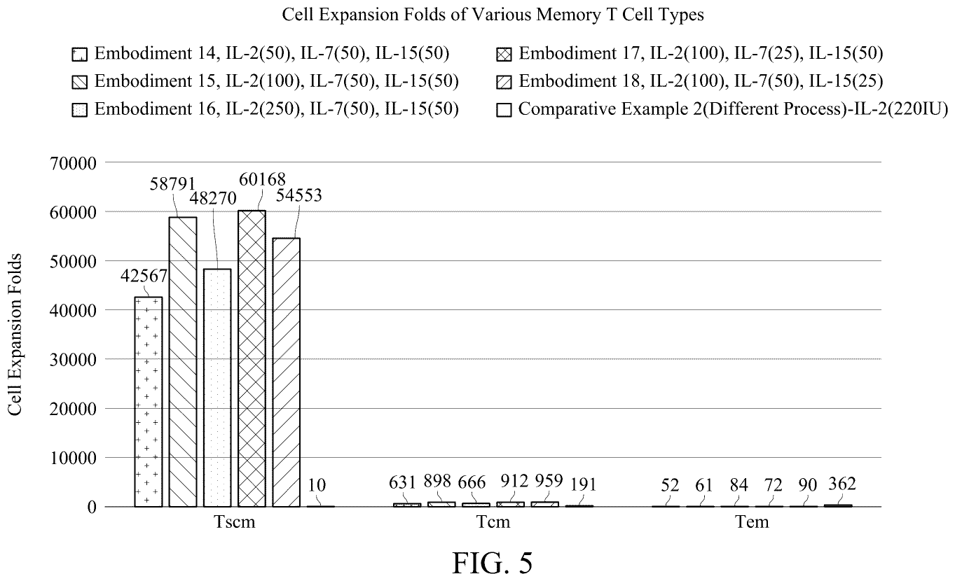

FIG. 5 and Table 8 show the cell expansion folds of the stem cell memory T cells (Tscm), the central memory T cells (Tcm), and the effector memory T cells (Tem) obtained using Embodiments 14 to 18 and Comparative Example 2.

FIG. 5 and Table 8 show that after culturing cells in a basal medium supplemented with the compositions of Embodiments 14 to 18, the results demonstrate that the stem cell memory T cells (Tscm), the central memory T cells (Tcm), and the effector memory T cells (Tem) all exhibit higher cell expansion folds compared to Comparative Example 2, wherein the cell expansion fold of the stem cell memory T cells (Tscm) is dramatically increased (greater than 10-fold) compared to Comparative Example 2.

TABLE 7
The concentrations of CD3, CD28, IL-2, IL-7, and IL-15 in the first composition,
the concentrations of IL-2, IL-7, and IL-15 in the second composition, and
the concentrations of NAC, hPL, IL-7 and IL-15 in the third composition

| | | Embodi- | Embodi- | | Embodi- | | Embodi- | | Embodi- | |
|---|---|---|---|---|---|---|---|---|---|---|
| | | ment 14 | ment 15 | | ment 16 | | ment 17 | | ment 18 | |
| First | CD3 | 200 ng/ml | | | | | | | | |
| Composition | CD28 | 600 ng/ml | | | | | | | | |
| | IL-2 | 50 U/ml | 100 | U/ml | 250 | U/ml | 100 | U/ml | 100 | U/ml |
| | IL-7 | 50 U/ml | 50 | U/ml | 50 | U/ml | 25 | U/ml | 50 | U/ml |
| | IL-15 | 50 U/ml | 50 | U/ml | 50 | U/ml | 50 | U/ml | 25 | U/ml |

| | | |
|---|---|---|
| Second | IL-2 | 100 U/ml |
| Composition | IL-7 | 50 U/ml |
| | IL-15 | 50 U/ml |
| Third | NAC | 10 mM |
| Composition | hPL | 5% |
| | IL-7 | 50 U/ml |
| | IL-15 | 50 U/ml |

TABLE 8
Cell expansion folds of various memory T cell types

| | Tscm | Tcm | Tem |
|---|---|---|---|
| Embodiment 14 | 42567 | 631 | 52 |
| Embodiment 15 | 58791 | 898 | 61 |
| Embodiment 16 | 48270 | 666 | 84 |
| Embodiment 17 | 60168 | 912 | 72 |
| Embodiment 18 | 54553 | 959 | 90 |
| Comparative Example 2 | 10 | 191 | 362 |

### [Experiment 5: Statistical Analysis of the Proportions of Activated T Cells, Natural Killer T Cells, and Cytotoxic T Cells in the Second Cell Population]

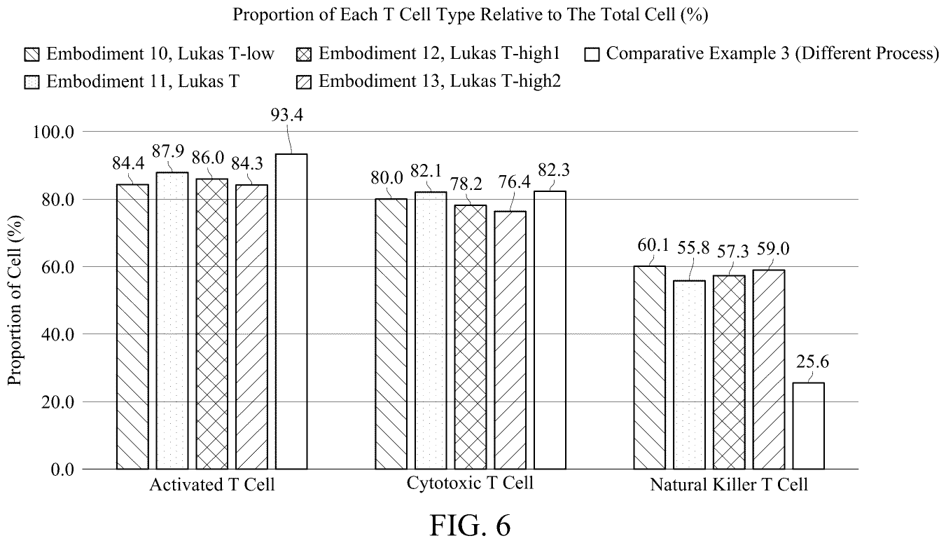

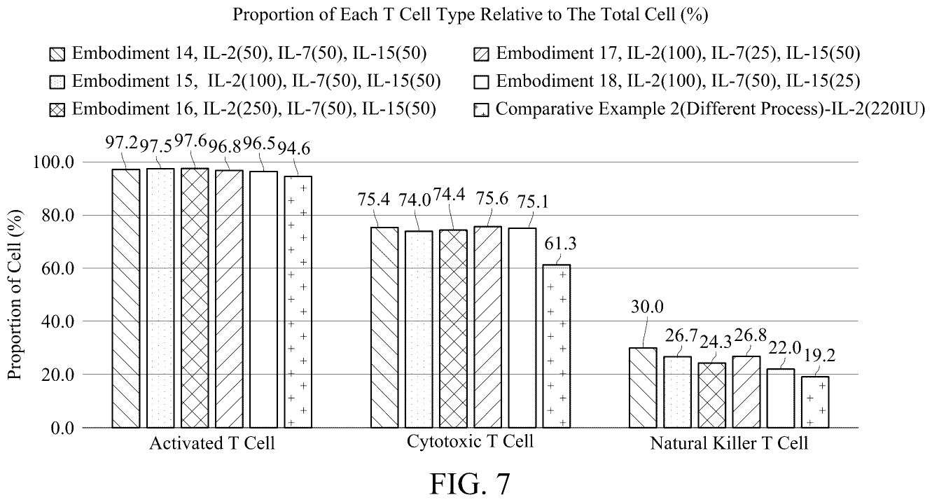

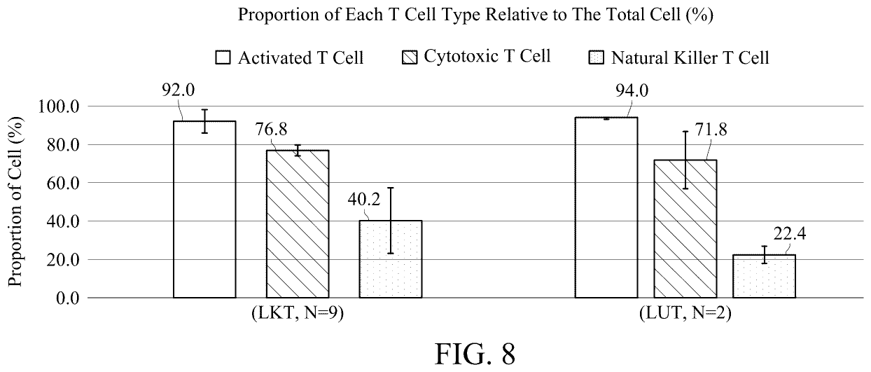

In this experiment, data from Embodiments 10 to 13 and Comparative Example 3, which evaluate the concentrations of CD3 and CD28 (see FIG. 6), and data from Embodiments 14 to 18 and Comparative Example 2, which evaluate the concentrations of IL-2, IL-7, and IL-15 (see FIG. 7), are collected to show the proportion of each T cell type relative to the total cell number for each batch, as illustrated in FIG. 8, FIG. 6 and FIG. 7 illustrate the statistical analysis of the proportions of CD3+ activated T cells, CD3+CD8+ cytotoxic T cells, and CD3+CD56+ natural killer T cells relative to the total cell number for each batch in the second cell population. As shown in FIGS. 6 and 7, the results show that using the compositions of the embodiments of the present disclosure yields high proportions of activated T cells, cytotoxic T cells, and natural killer T cells.

Comparative Example 3 which employs a two-stage cell culture method, is provided:
First stage (Day 0 to Day 5): mononuclear cells are cultured in X-Vivo basal medium supplemented with 50 μg/mL gentamicin, 5% human serum, 220 U/mL IL-2, and 5 μg/mL CD3 to obtain an intermediate cell product.
Second stage (Day 6 to Day 14): the intermediate cell product is further cultured in X-Vivo basal medium supplemented with 50 μg/mL gentamicin, 5% human serum, and 220 U/mL IL-2 to obtain the target cell population. The cell expansion fold of the target cell population is then calculated and analyzed.

Referring to FIG. 6, specifically, the proportion of activated T cells in the second cell population may be at least 84.3%, and more specifically, may be 84.3% to 87.9%. Specifically, the proportion of cytotoxic T cells in the second cell population may be at least 76.4%, and more specifically, may be 76.4% to 82.1%. Specifically, the proportion of natural killer T cells in the second cell population may be at least 55.8%, and more specifically, may be 55.8% to 60.1%, and higher than Comparative Example 3 (greater than 25.6%).

Referring to FIG. 7, specifically, the proportion of activated T cells in the second cell population may be at least 96.5%, and more specifically, may be 96.5% to 97.5%, and higher than Comparative Example 2 (greater than 94.6%). Specifically, the proportion of cytotoxic T cells in the second cell population may be at least 74.0%, and more specifically, may be 74.0% to 75.6%, and higher than Comparative Example 2 (greater than 61.3%). Specifically, the proportion of natural killer T cells in the second cell population may be at least 22.0%, and more specifically, may be 22.0% to 30.0%, and higher than Comparative Example 2 (greater than 19.2%).

FIG. 8, Table 9, and Table 10 show the proportions of various T cell types relative to the total cell number for each batch. The statistical results may further confirm that the second cell population obtained using the compositions of Embodiments 10 to 18 of the present disclosure has a significantly higher proportion of cytotoxic T cells (greater than 71.8%) and natural killer T cells (greater than 22.4%) compared to Comparative Examples 2 and 3.

TABLE 9
The proportion of each T cell type relative to the total cell
number for each batch obtained from Embodiments 10 to 18

| | Activated | Cytotoxic | Natural Killer |
|---|---|---|---|
| Group | T Cell | T Cell | T Cell |
| Embodiment 10 | 84.4 | 80.0 | 60.1 |
| Embodiment 11 | 87.9 | 82.1 | 55.8 |
| Embodiment 12 | 86.0 | 78.2 | 57.3 |
| Embodiment 13 | 84.3 | 76.4 | 59.0 |
| Embodiment 14 | 97.2 | 75.4 | 30.0 |
| Embodiment 15 | 97.5 | 74.0 | 26.7 |
| Embodiment 16 | 97.6 | 74.4 | 24.3 |
| Embodiment 17 | 96.8 | 75.6 | 26.8 |
| Embodiment 18 | 96.5 | 75.1 | 22.0 |
| Average | 92.0 | 76.8 | 40.2 |
| Standard Deviation | 6.1 | 2.8 | 17.1 |
| Maximum | 97.6 | 82.1 | 60.1 |
| Minimum | 84.3 | 74.0 | 22.0 |

TABLE 10
The proportion of each T cell type relative to the total cell number
for each batch obtained from Comparative Examples 2 and 3

| | Activated | Cytotoxic | Natural Killer |
|---|---|---|---|
| Group | T Cell | T Cell | T Cell |
| Comparative | 94.6 | 61.3 | 19.2 |
| Example 2 | | | |
| Comparative | 93.4 | 82.3 | 25.6 |
| Example 3 | | | |
| Average | 94.0 | 71.8 | 22.4 |
| Standard | 0.9 | 14.9 | 4.5 |
| Deviation | | | |
| Maximum | 94.6 | 82.3 | 25.6 |
| Minimum | 93.4 | 61.3 | 19.2 |

[Experiment 6. Comparison of the Cell Expansion Fold of Different Memory T Cells with Comparative Examples]

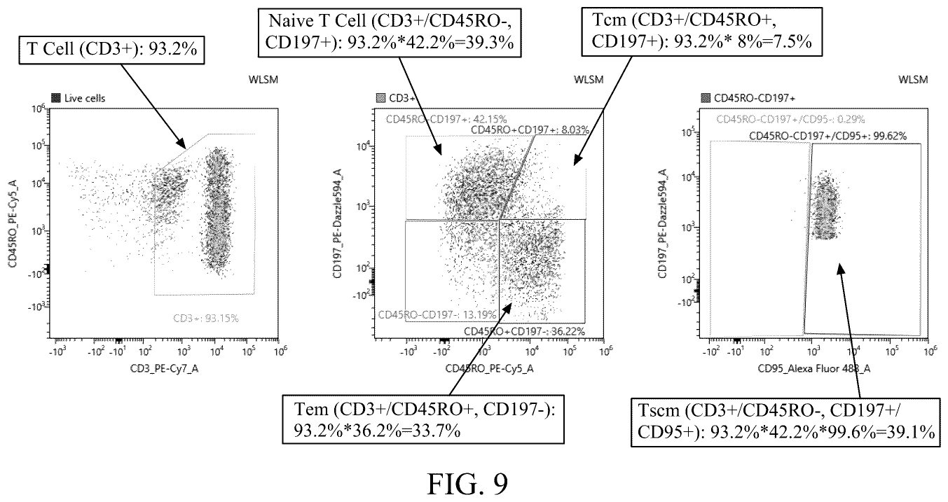

FIG. 9 is a diagram illustrating a flow cytometric analysis of the cells obtained by culturing using the first composition to the third composition of Embodiment 11. Table 11 shows the calculated cell expansion fold of different memory T cells based on the proportions obtained from the flow cytometry analysis.

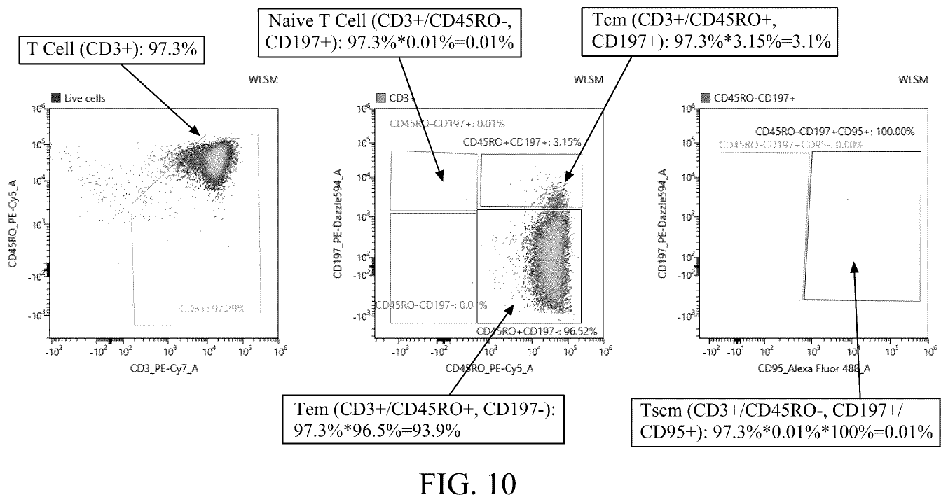

FIG. 10 is a diagram illustrating a flow cytometry analysis of the cells obtained by culturing using the process of Comparative Example 2. Table 12 shows the calculated cell expansion fold of different memory T cells based on the proportions obtained from the flow cytometry analysis.

TABLE 11
The proportion and the calculated cell expansion fold of the
different memory T cells obtained using Embodiment 11.

| | 446 × 10⁶ cells | | | |
|---|---|---|---|---|
| | | Cell | Number of Each Cell | |
| | | Expansion | Type on Day 0 | Number of Each Cell |
| Total Cell Number | Proportion | Fold | (Mononuclear Cells) | Type after Culture |
| T Cell | 93.2% | 427 | 9.7 × 10⁵ | 415.7 × 10⁶ |
| Tscm | 39.1% | 217225 | 8 × 10² | 174.4 × 10⁶ |
| Tcm | 7.5% | 1939 | 1.7 × 10⁴ | 33.4 × 10⁶ |
| Tem | 33.7% | 237 | 6.3 × 10⁵ | 150.5 × 10⁶ |

TABLE 12
The proportion and the calculated cell expansion fold of the different
memory T cells obtained using Comparative Example 2.

| | 327.1 × 10⁶ cells | | | |
|---|---|---|---|---|
| | | Cell | Number of Each Cell | |
| | | Expansion | Type on Day 0 | Number of Each Cell |
| Total Cell Numer | Proportion | Fold | (Mononuclear Cells) | Type after Culture |
| T Cell | 97.3% | 241 | 1.3 × 10⁶ | 318.3 × 10⁶ |
| Tscm | 0.01% | 10 | 3 × 10³ | 3.2 × 10⁴ |
| Tcm | 3.1% | 191 | 5.2 × 10⁴ | 10 × 10⁶ |
| Tem | 93.9% | 362 | 6.3 × 10⁵ | 150.5 × 10⁶ |

The experimental results indicate that using the first composition to the third composition with specific components and concentrations, as described in the present disclosure, effectively expands the cell expansion folds of both central memory cells and memory T cells. In particular, the cell expansion fold of stem cell memory T cells (Tscm) increases dramatically, exceeding a 10-fold increase compared to Comparative Example 2.

As described above, the composition and the method for ex vivo expansion of memory T stem cells of the present disclosure can significantly enhance the cell expansion fold of stem cell memory T cells while also achieving a higher expansion fold of central memory T cells. Furthermore, compared to the cell expansion folds achieved by compositions disclosed in the prior art, the expansion folds achieved by the first composition to the third composition of the present disclosure are more pronounced. This improvement increases the production efficiency of stem cell memory T cells and enhances their potential for use in cancer treatment or as a critical raw material in the manufacture of chimeric antigen receptor T cells (CAR-T cells).

It will be apparent to those skilled in the art that various modifications and variations can be made to the disclosed methods and materials. It is intended that the specification and examples be considered as exemplary only, with the true scope of the disclosure being indicated by the following claims and their equivalents.

What is claimed is:

1. A composition for ex vivo expansion of stem cell memory T cells, comprising:
a first composition comprising CD3, CD28, interleukin-2 (IL-2), interleukin-7 (IL-7) and interleukin-15 (IL-15); and
a second composition comprising interleukin-2, interleukin-7 and interleukin-15.

2. The composition for ex vivo expansion of stem cell memory T cells according to claim 1, further comprising:
a third composition comprising:
50 U/mL, 0.385 ng/mL, or 25 ng/mL of interleukin-7;
50 U/mL, 2.5 ng/mL, or 25 ng/ml of interleukin-15;
10 mM of N-acetylcysteine (NAC); and
5% (v/v) of human platelet lysate.

3. The composition for ex vivo expansion of stem cell memory T cells according to claim 1, wherein the first composition comprises:
100 ng/ml to 800 ng/ml of CD3;
300 ng/ml to 2000 ng/ml of CD28;
50 U/mL to 250 U/mL of interleukin-2 or 5 ng/ml to 41 ng/mL of interleukin-2;
25 U/mL to 50 U/mL of interleukin-7 or 0.192 ng/mL to 25 ng/mL of interleukin-7; and
10 U/mL to 50 U/mL of interleukin-15 or 0.5 ng/ml to 25 ng/ml of interleukin-15.

4. The composition for ex vivo expansion of stem cell memory T cells according to claim 1, wherein the first composition comprises:
100 ng/ml to 200 ng/ml of CD3;
300 ng/mL to 600 ng/ml of CD28;
50 U/mL to 250 U/mL of interleukin-2 or 5 ng/ml to 41 ng/ml of interleukin-2;
25 U/mL to 50 U/mL of interleukin-7 or 0.192 ng/mL to 25 ng/mL of interleukin-7; and
25 U/mL to 50 U/mL of interleukin-15 or 1.25 ng/ml to 25 ng/ml of interleukin-15.

5. The composition for ex vivo expansion of stem cell memory T cells according to claim 1, wherein the second composition comprises:
100 U/mL of interleukin-2 or 10 ng/ml or 16.4 ng/ml of interleukin-2;
50 U/mL of interleukin-7 or 0.385 ng/mL or 25 ng/ml of interleukin-7; and
50 U/mL of interleukin-15 or 2.5 ng/mL or 25 ng/ml of interleukin-15.

6. A method for ex vivo expansion of stem cell memory T cells, comprising:
culturing mononuclear cells in a medium containing a first composition, wherein the first composition comprising CD3, CD28, interleukin-2 (IL-2), interleukin-7 (IL-7) and interleukin-15 (IL-15) to obtain activated T cells;
culturing the activated T cells in a medium containing a second composition, wherein the second composition comprising interleukin-2, interleukin-7 and interleukin-15 to obtain a first cell population; and
culturing the first cell population in a medium containing a third composition, wherein the third composition comprising 50 U/mL, 0.385 ng/mL, or 25 ng/ml of interleukin-7, 50 U/mL, 2.5 ng/mL, or 25 ng/ml of interleukin-15, 10 mM of N-acetylcysteine (NAC), and 5% (v/v) of human platelet lysate to obtain a second cell population.

7. The method for ex vivo expansion of stem cell memory T cells according to claim 6, wherein the activated T cells are CD3+ activated T cells;
the first cell population comprises CD3+CD45RO−CD197+CD95+ stem cell memory T cells, CD3+CD45RO+CD197+ central memory T cells, CD3+CD45RO+CD197− effector memory T cells, CD3+CD56+ natural killer T cells, and CD3+CD8+ cytotoxic T cells; and
the second cell population comprises CD3+ activated T cells, CD3+CD45RO−CD197+CD95+ stem cell memory T, CD3+CD45RO+CD197+ central memory T cells, CD3+CD45RO+CD197− effector memory T cells, CD3+CD56+ natural killer T cells, and CD3+CD8+ cytotoxic T cells.

8. The method for ex vivo expansion of stem cell memory T cells according to claim 6, wherein a cell culture temperature is 37° C.

9. The method for ex vivo expansion of stem cell memory T cells according to claim 6, wherein the mononuclear cells are cultured for 2 to 4 days to obtain the activated T cells;
the activated T cells are cultured for 4 to 6 days to obtain the first cell population; and
the first cell population is cultured for 3 to 6 days to obtain the second cell population.
